(19) United States
(12) Patent Application Publication
(10) Pub. No.: US 2026/0306118 A1
(43) Pub. Date: Oct. 8, 2026

(54) CYCLIC ASPIRATION SYSTEM PRODUCING A CYCLIC ASPIRATION PRESSURE WAVEFORM HAVING A FLUID REPLENISHMENT SYSTEM

(71) Applicant: NEURAVI LIMITED, GALWAY (IE)

(72) Inventors: David VALE, Barna (IE); Alan CARNEY, Galway (IE)

(73) Assignee: NEURAVI LIMITED, GALWAY (IE)

(21) Appl. No.: 19/578,570

(22) Filed: Mar. 25, 2026

Related U.S. Application Data
(60) Provisional application No. 63/782,765, filed on Apr. 3, 2025.

(51) Int. Cl. A61B 17/22; A61B 17/00

(52) U.S. Cl. CPC A61B 17/22; A61B 2017/00154; A61B 2017/00477; A61B 2017/00544; A61B 2017/00561; A61B 2017/00778; A61B 2017/22079; A61B 2217/005

(57) ABSTRACT

A cyclic aspiration catheter includes an aspiration catheter, a pulsatile pressure and aspiration source, a supply tube and a fluid reservoir. The aspiration catheter has a proximal end and a distal end. The pulsatile pressure and aspiration source is in fluid communication with the proximal end of the catheter. The supply tube is disposed within the aspiration catheter. The supply tube has a proximal end and an open distal end. The open distal end is in fluid communication with the aspiration catheter. The fluid reservoir is in fluid communication with the supply tube such that a fluid from the reservoir can be drawn into the aspiration catheter when the pressure in the aspiration catheter is lower than that in the supply tube.

### CROSS REFERENCE TO RELATED APPLICATIONS

This application claims benefit of priority under 35 U.S.C. § 119 to prior filed U.S. Provisional Patent Application No. 63/782,765, filed Apr. 3, 2025 (Attorney Docket No.: 243382.000604(NRV6152USPSP1)), the entire contents of which is hereby incorporated by reference in its entirety as if set forth in full herein.

### FIELD

The present disclosure generally relates to a system and method used during thrombectomy procedures for the capture and removal of occlusions or clots. Specifically, the present disclosure relates to a cyclic aspiration system for the capture and removal of occlusions or clots in a vessel where the cyclic aspiration pressure waveform includes intermittent cyclic intervals of vacuum pressure (i.e., below atmospheric pressure) and positive pressure (i.e., higher than vacuum pressure, possibly higher than atmospheric pressure). The cyclic aspiration system produces the cyclic aspiration waveform using a positive pressure pulse generator mechanism associated with a conduit disposed in fluid communication between a vacuum pump and aspiration catheter, wherein the positive pressure pulse generator mechanism includes a fluid replenishment system to reduce the likelihood of air bubbles from entering the vasculature.

### BACKGROUND

Pulsatile or cyclic aspiration applies a cyclic pressure waveform of intermittent cyclic minimum/low/vacuum/aspiration pressure and maximum/peak/high pressure. During cycles under the minimum/low/vacuum/aspiration pressure the clot is drawn in the proximal direction and captured at the distal tip/end of the aspiration catheter, whereas during cycles of maximum/peak/high pressure the clot is pushed in the distal direction. When using pulsatile or cyclic aspiration for clot capture and removal, it is important to reduce the likelihood of air bubbles from entering the vasculature, particularly during the high-pressure phase of the cyclic aspiration cycle.

It is therefore desirable to develop an improved cyclic aspiration system utilizing a mechanism that ensures the integrity of the aspiration system, particularly during the high-pressure phase of the cyclic aspiration cycle while reduce the likelihood of air bubbles from entering the vasculature. Still further desirable is to develop an improved cyclic aspiration system that minimizes a risk of air bubbles from entering the vasculature.

### SUMMARY

An aspect of the present disclosure relates to an aspiration catheter comprising a proximal end and a distal end; a pulsatile pressure and aspiration source being in fluid communication with the proximal end of the catheter; a supply tube disposed within the aspiration catheter, the supply tube having a proximal end and an open distal end, the open distal end being in fluid communication with the aspiration catheter; and a fluid reservoir being in fluid communication with the supply tube such that a fluid from the reservoir can be drawn into the aspiration catheter.

Another aspect of the present disclosure is directed to a cyclic aspiration catheter comprising a first catheter having a proximal end and an open distal end; a second catheter having a proximal and an open distal end; a pulsatile pressure and aspiration source being in fluid communication with the proximal end of the first catheter; a fluid reservoir being in fluid communication with proximal end of the second tube; and a third catheter having a proximal end and an open distal end.

Another aspect of the present disclosure is directed to a method of reducing a risk of air bubbles from entering the vasculature when operation a cyclic aspiration catheter to capture and remove an occlusion within the vessel. The method comprises tracking the aspiration catheter to a site within the vasculature adjacent to an occlusion within the vasculature; applying an intermittent cyclic interval of vacuum pressure and positive pressure within the aspiration catheter to capture and remove the occlusion; delivering a liquid from the supply tube within the aspiration catheter during a positive pressure portion of the intermittent cyclic interval; and removing an air bubble from the aspiration catheter via the proximal end of the aspiration catheter during a vacuum pressure portion of the intermittent cyclic interval.

Another aspect of the present disclosure is directed to a method of reducing a risk of air bubbles from entering the vasculature when operation a cyclic aspiration catheter to capture and remove an occlusion within the vessel, the method comprises tracking the aspiration catheter to a site within the vasculature adjacent to an occlusion within the vasculature; applying an intermittent cyclic interval of vacuum pressure and positive pressure from the first catheter within the aspiration catheter to capture and remove the occlusion; delivering a liquid from the second catheter within the aspiration catheter during a positive pressure portion of the intermittent cyclic interval; and removing an air bubble from the aspiration catheter via a proximal end of the first catheter during a vacuum pressure portion of the intermittent cyclic interval.

Another aspect of the present disclosure relates to a pulsatile or cyclic aspiration system producing a cyclic aspiration pressure waveform of intermittent cyclic intervals of vacuum pressure below atmospheric pressure and positive pressure higher than vacuum pressure (higher than vacuum pressure, possibly higher than atmospheric pressure) using as few active components as possible.

Another aspect of the present disclosure is directed to a cyclic aspiration system producing a cyclic aspiration pressure waveform using a vacuum pump connected in fluid communication with an aspiration catheter via a conduit (e.g., inlet tubing, housing, or a rotating hemostatic valve) having a positive pressure pulse generator mechanism and associated at least one gating device.

Another aspect of the present disclosure is directed to a cyclic aspiration system producing a cyclic aspiration pressure waveform using a pulsatile vacuum pump connected in fluid communication with an aspiration catheter via a conduit (e.g., inlet tubing, housing, or a rotating hemostatic valve) and a fluid flush bag in fluid communication with the aspiration catheter.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices, by way of example only, not by way of limitation.

FIG. 1A is a graph illustrates the amount of cyclic pressure/aspiration on the y-axis applied over time on the x-axis being applied to a cyclic aspiration catheter during an aspiration portion of the cycle;

FIG. 1B is a partial cross-sectional view of the aspiration catheter of FIG. 1A having aspiration applied to the catheter;

FIG. 2A is a graph illustrates the amount of cyclic pressure/aspiration on the y-axis applied over time on the x-axis being applied to a cyclic aspiration catheter during a pressure portion of the cycle;

FIG. 2B is a partial cross-sectional view of an aspiration catheter of FIG. 2A having pressure applied to the catheter;

FIG. 3A is a graph illustrates the amount of cyclic pressure/aspiration on the y-axis applied over time on the x-axis being applied to a cyclic aspiration catheter during an aspiration portion of the cycle;

FIG. 3B is a partial cross-sectional view of the cyclic aspiration catheter of FIG. 3A having aspiration applied to the catheter;

FIG. 4A is a graph illustrates the amount of cyclic pressure/aspiration on the y-axis applied over time on the x-axis being applied to a cyclic aspiration catheter during a pressure portion of the cycle;

FIG. 4B is a partial cross-sectional view of the aspiration catheter of FIG. 4A having pressure applied to the cyclic aspiration catheter;

FIG. 5 is a partial cross-sectional view of a cyclic aspiration catheter having a flushing fluid source and a pulsatile cyclic pressure/aspiration source connected to a proximal end of the catheter;

FIG. 6 is a partial cross-sectional view of a cyclic aspiration catheter having a flushing fluid source and a pulsatile cyclic pressure/aspiration source connected to a proximal end of the catheter;

FIG. 7A is a graph illustrates the amount of cyclic pressure/aspiration on the y-axis applied over time on the x-axis being applied to a cyclic aspiration catheter during an aspiration portion of the cycle;

FIG. 7B is a partial cross-sectional view of the aspiration catheter of FIG. 7A having aspiration applied to the cyclic aspiration catheter and a flushing fluid source being connected to a proximal end of the catheter;

FIG. 8A is a graph illustrates the amount of cyclic pressure/aspiration on the y-axis applied over time on the x-axis being applied to a cyclic aspiration catheter during a pressure portion of the cycle;

FIG. 8B is a partial cross-sectional view of the aspiration catheter of FIG. 8A having pressure applied to the cyclic aspiration catheter and a flushing fluid source being connected to a proximal end of the catheter;

FIG. 9A is a graph illustrates the amount of cyclic pressure/aspiration on the y-axis applied over time on the x-axis being applied to a cyclic aspiration catheter during an aspiration portion of the cycle;

FIG. 9B is a partial cross-sectional view of the aspiration catheter of FIG. 9A having aspiration applied to the cyclic aspiration catheter and a flushing fluid source being connected to a proximal end of the catheter;

FIG. 10A is a graph illustrates the amount of cyclic pressure/aspiration on the y-axis applied over time on the x-axis being applied to a cyclic aspiration catheter during an aspiration portion of the cycle;

FIG. 10B is a partial cross-sectional view of the aspiration catheter of FIG. 10A having aspiration applied to the cyclic aspiration catheter and a flushing fluid source being connected to a proximal end of the catheter;

FIG. 11A is a partial cross-sectional view of an aspiration catheter having aspiration applied to the cyclic aspiration catheter and a flushing fluid source being connected to a proximal end of the catheter;

FIG. 11B is a partial cross-sectional view of the aspiration catheter having aspiration applied to the cyclic aspiration catheter and a flushing fluid source being connected to a proximal end of the catheter; and

FIG. 12 is a partial cross-sectional view of a cyclic aspiration catheter having a flushing fluid source and a pulsatile cyclic pressure/aspiration source connected to a proximal end of the catheter;

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g., “about 90%” may refer to the range of values from 81% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

The present disclosure is directed to a cyclic aspiration system for producing a cyclic aspiration pressure waveform of intermittent cyclic intervals of vacuum pressure below atmospheric pressure and positive pressure higher than vacuum pressure, possibly higher than atmospheric pressure, using a vacuum pump connected in fluid communication to the proximal end of an aspiration catheter via a conduit with an associated positive pressure pulse generator mechanism for intermittently cyclically producing the positive pressure pulse. The conduit may include: (i) inlet tubing (e.g., vacuum inlet tubing and/or positive pressure inlet tubing) disposed between the vacuum pump and the aspiration catheter; or (iii) a rotating hemostatic valve (RHV) connected to the aspiration catheter. Numerous non-limiting examples of various cyclic aspiration system for creating the cyclic aspiration system using a vacuum pump can be used within the scope of the present disclosure.

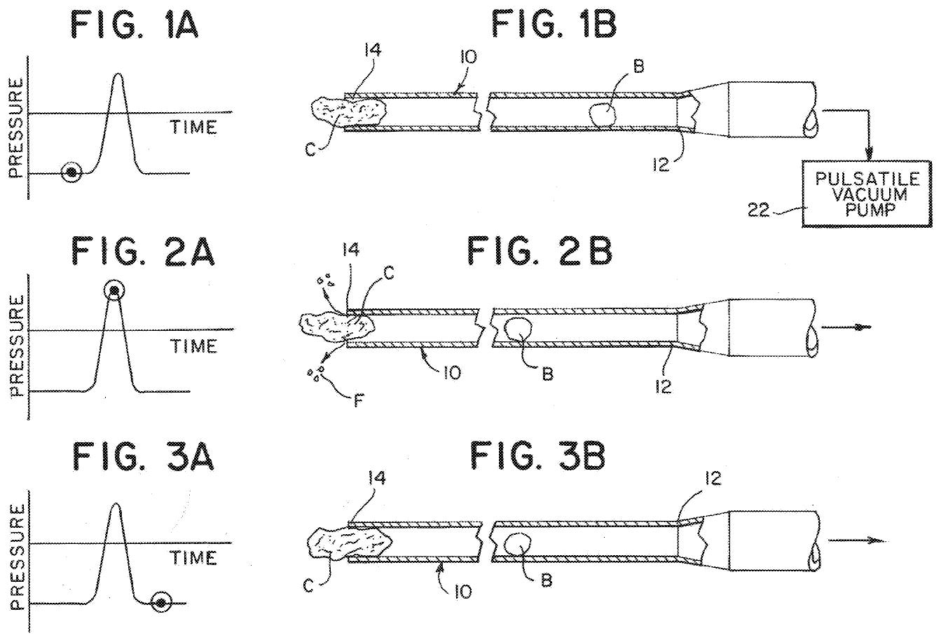

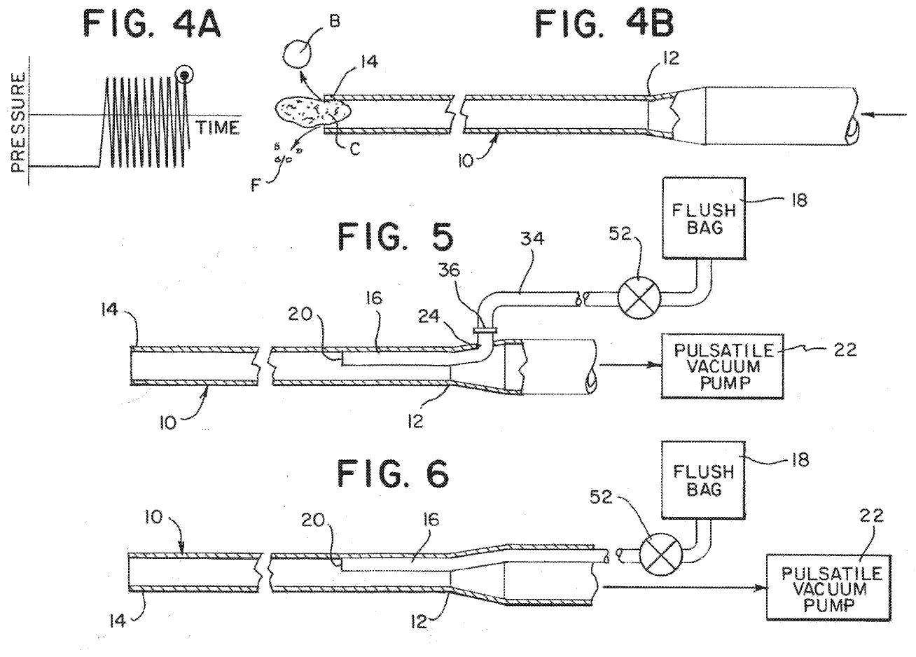

Referring now to FIGS. 1A-4, an aspiration catheter 10 having a proximal end 12 and a distal end 14. Aspiration catheter 10 is navigated in the vasculature of a patient to try and remove an occlusion such as, for example, a blood clot C from a blood vessel (not shown). A pulsatile pressure/aspiration source or pulse generator 22 is in fluid communication with the proximal end 12 of catheter 10. Clot C is engaged with the distal end 14 of catheter 10 due to the aspiration pressure applied from the pressure/aspiration source 22. An air bubble B is illustrated within catheter 10. As shown in FIG. 2B, as positive pressure is applied to the catheter, bubble B may travel distally within catheter 10. Pulsatile aspiration is most effective when the peak amplitude of the pulse is high enough to push the clot slightly forward (distal) in the catheter tip, before the next vacuum phase pulls it back into the catheter tip, as shown, for example, in FIG. 2B. Clot ingestion is significantly enhanced when this pulse amplitude is sufficient to allow some of the fluid F within the catheter tip to escape distally past the clot into the vasculature on each positive pressure pulse, or at least on some positive pressure pulses, as shown in FIG. 2B. This loss of fluid distally means that the contents of the aspiration catheter can gradually migrate towards the distal end of the catheter during pulsatile aspiration with a clot lodged in the distal catheter tip. This may not be a problem if the catheter is filled with a fluid such as saline but could result in patient harm if there is any air, such as air bubble B, in the catheter. This air could be present in the catheter if the user has not kept the catheter flushed during its advancement to the clot and during subsequent removal of any inner devices such as access aids/microcatheter/guidewires, etc. Air could also be introduced during connection of the aspiration catheter hub to the pulsatile pump connection tubing. Air could even be present in the connection tubing itself if it is not prepped properly prior to use. Ensuring the system is prepped and used carefully so that no air is in the system is possible but relies on the user to exercise considerable care during the case. As shown in FIG. 4B, saline F and an air bubble B could be moved distally within catheter 10 and exit out of the distal end 14 of the catheter 10 past clot C and into the vasculature. It would be very desirable if the pulsatile system could be designed so that it was safe even in scenarios where some air was present in the system.

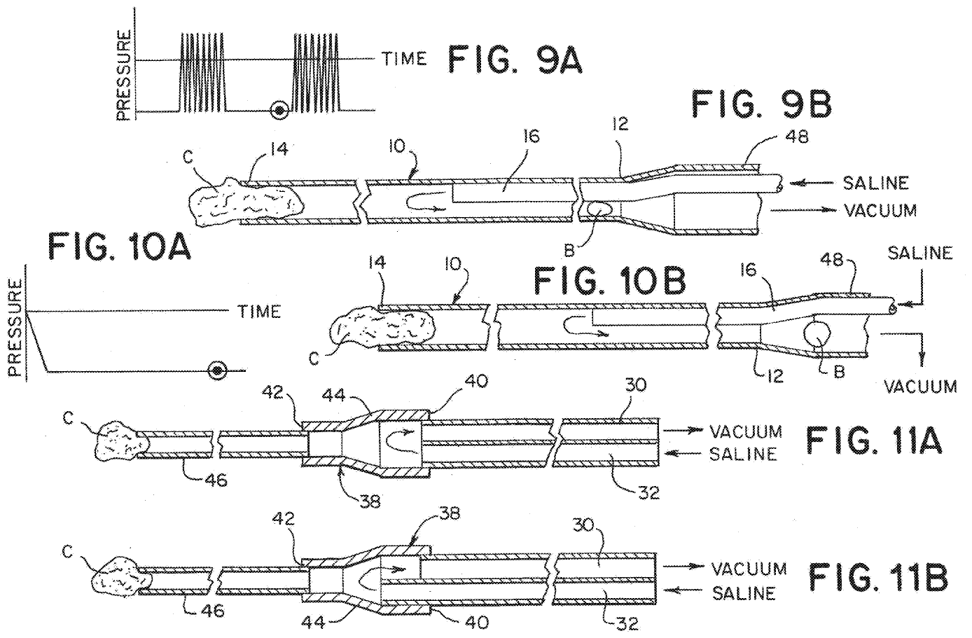

Referring now to FIGS. 5-11B, one solution in accordance with the present disclosure, is to counterbalance the distal leakage of fluid past the clot with a supply of fluid delivered to a point within the aspiration catheter that is distal to any entrained air, but proximal of the distal catheter tip. In FIGS. 8-10B, this is achieved by providing a “catheter within a catheter.” For example, a supply tube 16 or a lumen is provided within the aspiration catheter 10, which is connected proximally to a fluid reservoir 18, and is open at its distal end 20 so that the fluid from the reservoir 18 (flush bag in FIG. 5) can be drawn into the aspiration catheter 10 if the pressure in the aspiration catheter is lower than that in the supply tube 16. Fluid reservoir 18 could be pressurized, or non-pressurized. For example, fluid reservoir can be pressurized by gravity, simply by raising its height, including when reservoir 18 is in the form of a flush bag, above the height of the supply tube 16.

When the pulsatile pump is working, the pressure within the aspiration catheter cycles between high and low levels. During a positive pressure pulse, with a clot C lodged in the catheter tip, some fluid F may leak past the clot C from the aspiration catheter. After a series of such pulses a column of fluid will then have moved distally in the aspiration catheter, potentially moving any air in the catheter closer to the distal tip. The system then switches into a steady state vacuum mode for a predetermined number of seconds, during which time fluid is drawn from the supply tube into the lower pressure region within the aspiration catheter 10. The dwell time in this vacuum state and the length and cross-sectional area of the supply tube are configured such that the volume of fluid that is drawn from the supply tube in this phase is similar to, or ideally slightly greater than, the volume of fluid that leaked past the clot in the pulsatile phase. Thus, a column of fluid proximal to the distal end of the supply tube will move proximally in the catheter, away from the distal tip 14, which in turn will carry any air bubbles B in that portion of the catheter proximally with column of fluid.

An optional valve 52 is shown, which may be closed to isolate the reservoir from the supply tube during positive pressure pulses and opened during the vacuum dwell phases. This system could also be used to flush and expel air from the aspiration catheter and connection tubing prior to initiating pulsatile aspiration. To do this the aspiration catheter would be pushed against the clot and vacuum applied with valve open, driving any trapped air proximally out of the catheter, through the connection tubing towards the pulsatile pressure/aspiration source 22. Alternatively, valve 52 could be closed during the vacuum dwell phases, to avoid any fluid influx through the supply tube 34 from damping the pulse.

FIG. 5 shows connection tubing 16 being integral with catheter 10. Reservoir 18 has a connecter tube 34 and optional valve 52 that can be selectively fluidly connected to fluid supply tube 16 at connection coupling 36 located at the catheter hub 24. Thus, fluid supply tube 16 can be selectively connected to reservoir 18 at catheter hub 24 as shown in FIG. 5. Alternatively, as shown in FIG. 6, the connection tubing 16 can be integral with the flush bag/reservoir 18 and optional valve 52. In this embodiment, the connection tube 16 and reservoir 18 can be selectively fluidly connected to catheter 10.

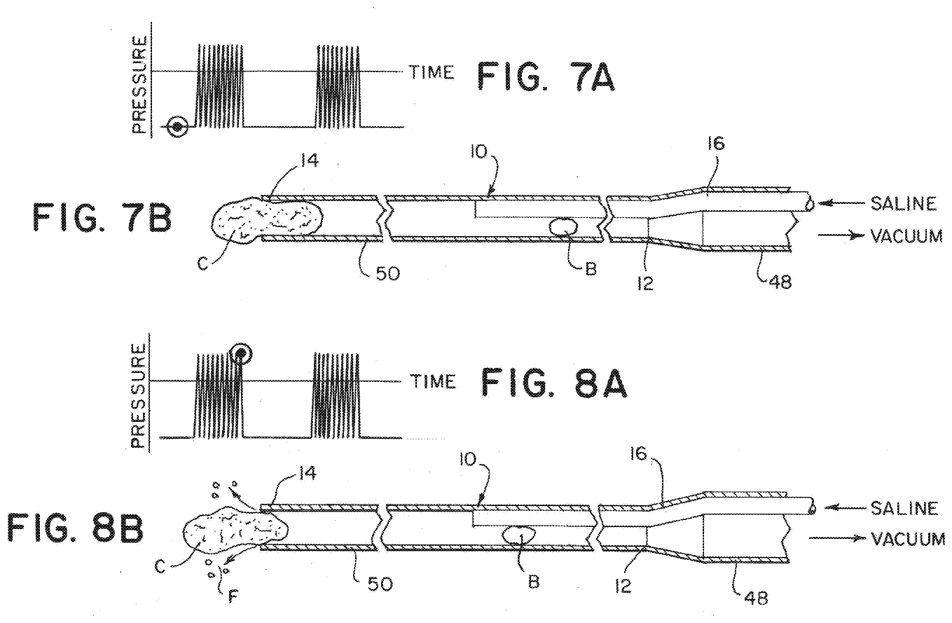

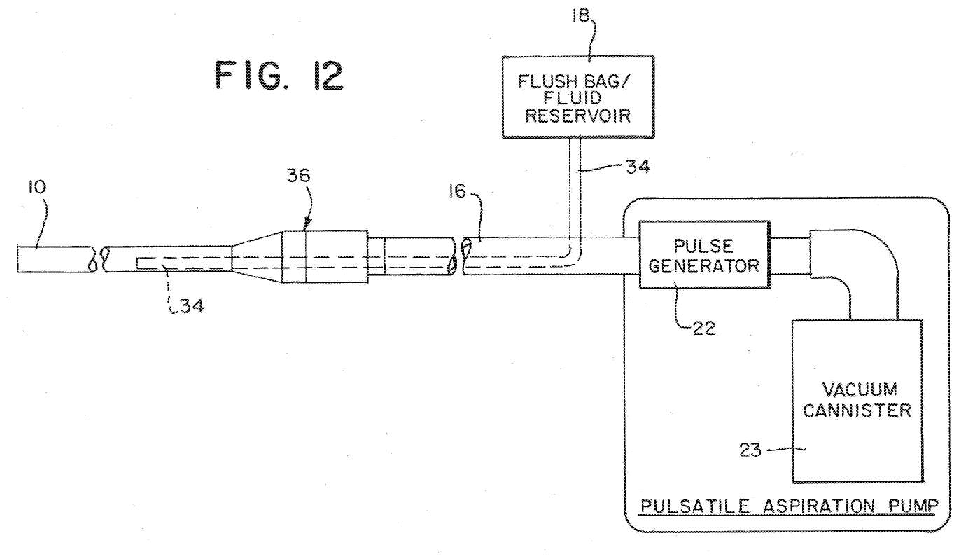

In FIGS. 7A and 7B, aspiration is being applied, and clot C is lodged in catheter tip at the distal end 14 of catheter 10. An air bubble B is illustrated as being present in the catheter lumen. In FIGS. 8A and 8B, pulsatile pressure causes fluid F to leak past clot C and air bubble B to move distally. In FIGS. 9A and 9B, during an aspiration dwell period, while in a vacuum state, fluid is drawn from supply tube 16 into catheter 10, and air bubble B moves proximally. In FIGS. 10A and 10B, a continuous vacuum could be applied to prepare and/or prime the system to remove any air prior to initiation of pulsatile aspiration.

If the supply tube 16 is integral to the catheter 10, supply tube 16 could be floating within the catheter lumen but connected to the catheter at the proximal hub connector of catheter 10. Alternatively, supply tube 16 could be incorporated into the wall of the catheter 10, or supply tube 16 could be attached to the inner wall of the catheter. Supply tube 16 can be circular in cross-section, including when supply tube 10 is floating within catheter 10. Supply tube 16 could also be more elliptical in cross-section or crescent-shaped if incorporated into the wall of the catheter.

Supply tube 16 and a connection tube 48 could be a fixed assembly, such that a user can slide supply tube 16 in the distal direction within the lumen of the narrow distal portion of connection tube or catheter 48. Supply tube 16 can be advanced until the hub of the connection tube 48 can be attached to a hub of supply tube 16. In an alternative aspect of the disclosure, supply tube 16 can be built into connection tube 48, which connects to another supply tube (not shown) built into the connection tube 48. In another aspect of the disclosure, supply tube 16 can be built into connection tube 48, which connects to another supply tube (not shown) external to the patient, which is in turn connected to the flush reservoir/bag 18.

Referring now to FIGS. 11A and 11B, in accordance with another aspect of the present disclosure, a one-way cyclic dual lumen 30, 32 embodiment is illustrated. In accordance with this aspect, the cyclic device will extract any air bubbles B after the connection is made by gently replacing any air bubbles B with saline via a second catheter 32 and vacuuming the air bubbles B out to the vacuum pump via a first or vacuum/aspiration catheter 30 simultaneously. A connector 38 has a proximal end 40 and a distal end 42. The proximal end 40 of connector 38 is in fluid communication with the distal end of the first catheter 30 and the distal end of the second catheter 32. The distal end 42 of connector 38 is in fluid communication with the proximal end of the third catheter 46. Connector 38 has a taper section 44 between its proximal end 40 and its distal end 42. Taper section 44 tapers radially inwardly in the distal direction from a proximal end of the taper section to a distal end of the taper section. In accordance with one aspect of the disclosure, the open distal end of the first catheter 30 and the open distal end of the second catheter 32 terminate at approximately the same axial location, as shown in FIG. 11A. The open distal end of second catheter 32 terminates distally of the open distal end of the first catheter 30, as shown in FIG. 11B. In accordance with another aspect of the disclosure, the open distal end of the first catheter 30 and the open distal end of the second catheter 32 are disposed within connector 38.

As shown in FIG. 11A, the distal end of each catheter terminates at the same axial location. In FIG. 11B, the distal end of the saline catheter 32 is more distal than the distal end of the vacuum/aspiration catheter 30. The saline catheter 32 has its distal end more distal than the source of the vacuum and pressure source, such as lumen 30 as shown in FIG. 11B.

FIG. 12 illustrates another aspect of the present disclosure. FIG. 12 shows connection tubing 16 being detachably connected to aspiration catheter 10 via a proximal catheter hub 24. Reservoir 18 has a supply tube 34 and optional valve 52 (not shown in FIG. 12) that can be selectively fluidly connected to fluid connection tube 16 and the aspiration catheter 10. Fluid connection tube 16 is fluidly connected to a vacuum cannister 23 and a pulse generator 22 as shown in FIG. 12.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

Aspects of the present disclosure are also provided by the following numbered Clauses:
Clause 1 A cyclic aspiration catheter comprising:
an aspiration catheter having a proximal end and a distal end;
a pulsatile pressure and aspiration source being in fluid communication with the proximal end of the catheter;
a supply tube disposed within the aspiration catheter, the supply tube having a proximal end and an open distal end, the open distal end being in fluid communication with the aspiration catheter; and
a fluid reservoir being in fluid communication with the supply tube such that a fluid from the reservoir can be drawn into the aspiration catheter.
Clause 2. The cyclic aspiration catheter of clause 1, the fluid reservoir being in fluid communication the proximal end of the supply tube.
Clause 3. The cyclic aspiration catheter of any of clauses 1-2, further comprising a valve fluidly connected to the supply tube, the valve being disposed between the proximal end of the supply tube and the open distal end of the supply tube.
Clause 4. The cyclic aspiration catheter of any of clauses 1-3, a distal portion of the supply tube being disposed within the aspiration catheter.
Clause 5. The cyclic aspiration catheter of any of clauses 1-4, the distal end of the supply tube being spaced proximally from the distal end of the aspiration catheter by a predetermined distance.
Clause 6. The cyclic aspiration catheter of any of clauses 1-5, fluid from the reservoir being drawn into the aspiration catheter when the pressure in the aspiration catheter is lower than that in the supply tube.
Clause 7. A cyclic aspiration catheter comprising:
a first tube having a proximal end and an open distal end;
a second tube having a proximal and an open distal end;
a pulsatile pressure and aspiration source being in fluid communication with the proximal end of the first catheter;
a fluid reservoir being in fluid communication with proximal end of the second tube; and
a first catheter having a proximal end and an open distal end.
Clause 8. The cyclic aspiration catheter of clause 7, further comprising a connector having a proximal end and a distal end; the proximal end of the connector being in fluid communication with the distal end of the first tube and the distal end of the second tube; the distal end of the connector being in fluid communication with the proximal end of the first catheter.
Clause 9. The cyclic aspiration catheter of any of clauses 7-8, the connector having a taper section between its proximal end and its distal end.
Clause 10. The cyclic aspiration catheter of any of clauses 7-9, the taper section tapering radially inwardly in the distal direction from a proximal end of the taper section to a distal end of the taper section.
Clause 11. The cyclic aspiration catheter of any of clauses 7-10, the open distal end of the first tube terminating distally of the open distal end of the second catheter.
Clause 12. The cyclic aspiration catheter of any of clauses 7-10, the open distal end of the first tube and the open distal end of the second tube terminate at approximately the same axial location.
Clause 13. The cyclic aspiration catheter of any of clauses 11-12, the open distal end of the first tube and the open distal end of the second tube being disposed within the connector.
Clause 14. A method of reducing a risk of air bubbles from entering the vasculature when operation a cyclic aspiration catheter to capture and remove an occlusion within the vessel, the method comprises an aspiration catheter having a proximal end and a distal end; a pulsatile pressure and aspiration source being in fluid communication with the proximal end of the catheter; a supply tube disposed within the aspiration catheter, the supply tube having a proximal end and an open distal end, the open distal end being in fluid communication with the aspiration catheter; and a fluid reservoir being in fluid communication with the supply tube such that a fluid from the reservoir can be drawn into the aspiration catheter, the method comprising:
tracking the aspiration catheter to a site within the vasculature adjacent to an occlusion within the vasculature;
applying an intermittent cyclic interval of vacuum pressure and positive pressure within the aspiration catheter to capture and remove the occlusion;
delivering a liquid from the supply tube within the aspiration catheter during a vacuum pressure portion of the intermittent cyclic interval; and
removing an air bubble from the aspiration catheter via the proximal end of the aspiration catheter during a vacuum pressure portion of the intermittent cyclic interval.
Clause 15. The method of reducing a risk of air bubbles from entering the vasculature of clause 14, capturing the occlusion at the distal end of the aspiration catheter while applying a vacuum pressure.
Clause 16. The method of reducing a risk of air bubbles from entering the vasculature of clause 15, applying a peak amplitude of the positive pressure that is sufficiently high to push the occlusion in the distal direction in the distal end of the aspiration catheter while maintaining at least a portion of the occlusion within the aspiration catheter.
Clause 17. The method of reducing a risk of air bubbles from entering the vasculature of clause 16, permitting liquid within the catheter to escape distally past the occlusion into the vasculature on a positive pressure pulse.
Clause 18. The method of reducing a risk of air bubbles from entering the vasculature of clause 17, permitting liquid within the catheter to escape distally past the occlusion into the vasculature on every positive pressure pulse.
Clause 19. The method of reducing a risk of air bubbles from entering the vasculature of any of clauses 17-18, after permitting liquid within the catheter to escape distally past the occlusion into the vasculature on a positive pressure pulse, applying a vacuum pressure to pull the occlusion proximally within the aspiration catheter.
Clause 20. The method of reducing a risk of air bubbles from entering the vasculature of any of clauses 14-19, the delivering a liquid from the supply tube includes delivering the liquid within the aspiration catheter at the open distal end of the supply tube, the distal end of the supply tube being spaced proximally from the distal end of the aspiration catheter by a predetermined distance.
Clause 21. A method of reducing a risk of air bubbles from entering the vasculature when operation a cyclic aspiration catheter to capture and remove an occlusion within the vessel, the method comprises an aspiration catheter, a connection tube having a proximal end and an open distal end; a supply tube having a proximal and an open distal end; a pulsatile pressure and aspiration source being in fluid communication with the proximal end of the connection tube; a fluid reservoir being in fluid communication with proximal end of the supply tube; and
a aspiration catheter having a proximal end and an open distal end, the method comprising:
   tracking the aspiration catheter to a site within the vasculature adjacent to an occlusion within the vasculature;
   applying an intermittent cyclic interval of vacuum pressure and positive pressure from the connection tube within the aspiration catheter to capture and remove the occlusion;
   delivering a liquid from the supply tube within the aspiration catheter during a positive pressure portion of the intermittent cyclic interval; and
   removing an air bubble from the aspiration catheter via a proximal end of the connection tube during a vacuum pressure portion of the intermittent cyclic interval.
Clause 22. The method of reducing a risk of air bubbles from entering the vasculature of clause 21, capturing the occlusion at the distal end of the aspiration catheter while applying a vacuum pressure.
Clause 23. The method of reducing a risk of air bubbles from entering the vasculature of clause 21, applying a peak amplitude of the positive pressure that is sufficiently high to push the occlusion in the distal direction in the open distal end of the aspiration catheter while maintaining at least a portion of the occlusion within the open distal end of the aspiration catheter.
Clause 24. The method of reducing a risk of air bubbles from entering the vasculature of clause 22, permitting liquid within the aspiration catheter to escape distally past the occlusion into the vasculature on a positive pressure pulse.
Clause 25. The method of reducing a risk of air bubbles from entering the vasculature of clause 24, permitting liquid within the aspiration catheter to escape distally past the occlusion into the vasculature on every positive pressure pulse.
Clause 26. The method of reducing a risk of air bubbles from entering the vasculature of any of clauses 24-25, after permitting liquid within the aspiration catheter to escape distally past the occlusion into the vasculature on a positive pressure pulse, applying a vacuum pressure to pull the occlusion proximally within the aspiration catheter.
Clause 27. The method of reducing a risk of air bubbles from entering the vasculature of any of clauses 21-26, the delivering a liquid from the supply tube includes delivering the liquid within the aspiration catheter at the open distal end of the supply tube, the distal end of the supply tube being spaced proximally from the distal end of the aspiration catheter by a predetermined distance.
Clause 28. The cyclic aspiration catheter of any of clauses 1-13, the fluid reservoir being pressurized.
Clause 29. The cyclic aspiration catheter of clause 28, the fluid reservoir being pressurized by raising its height above the supply tube.
Clause 30. The cyclic aspiration catheter of any of clauses 1-13, the fluid reservoir being non-pressurized.
Clause 31. The cyclic aspiration catheter of any of clauses 1-3, the valve being closed to isolate the fluid reservoir from the supply tube during positive pressure pulses and the valve being opened during the vacuum dwell phases.
Clause 32. The cyclic aspiration catheter of any of clauses 1-3, the valve being closed during the vacuum dwell phases, to avoid any fluid influx through the supply tube from damping a vacuum pulse.

The descriptions contained herein are examples and are not intended in any way to limit the scope of the present disclosure. As described herein, the present disclosure contemplates many variations and modifications of a pulsatile or cyclic aspiration system producing a cyclic aspiration pressure waveform of intermittent cyclic intervals of vacuum pressure below atmospheric pressure and positive pressure higher than vacuum pressure (higher than vacuum pressure, possibly higher than atmospheric pressure) using as few active components as possible with an associated maximized response time to attain maximum cycling frequency while also minimizing dampening or decay of the positive pressure wave as well as the additional benefit of reducing the overall cost of manufacture. The present disclosure further contemplates many variations and modifications of a cyclic aspiration system producing a cyclic aspiration pressure waveform using a vacuum pump connected in fluid communication with an aspiration catheter via a conduit (e.g., inlet tubing, housing, or a rotating hemostatic valve) having a positive pressure pulse generator mechanism and associated at least one gating device, wherein the at least one gating device includes at least one actuator component arranged externally of the conduit, not contaminated by blood, reusable, and separable from non-actuator components (e.g., conduit and components disposed therein) contaminated by blood and discardable after a single use. Modifications and variations apparent to those having skilled in the pertinent art according to the teachings of this disclosure are intended to be within the scope of the claims which follow.

What is claimed is:

1. A cyclic aspiration catheter comprising:
an aspiration catheter having a proximal end and a distal end;
a pulsatile pressure and aspiration source being in fluid communication with the proximal end of the catheter;
a supply tube disposed within the aspiration catheter, the supply tube having a proximal end and an open distal end, the open distal end being in fluid communication with the aspiration catheter; and
a fluid reservoir being in fluid communication with the supply tube such that a fluid from the reservoir can be drawn into the aspiration catheter.

2. The cyclic aspiration catheter of claim 1, the fluid reservoir being in fluid communication the proximal end of the supply tube.

3. The cyclic aspiration catheter of claim 2, further comprising a valve fluidly connected to the supply tube, the valve being disposed between the proximal end of the supply tube and the open distal end of the supply tube.

4. The cyclic aspiration catheter of claim 3, a distal portion of the supply tube being disposed within the aspiration catheter.

5. The cyclic aspiration catheter of claim 4, the distal end of the supply tube being spaced proximally from the distal end of the aspiration catheter by a predetermined distance.

6. The cyclic aspiration catheter of claim 5, fluid from the reservoir being drawn into the aspiration catheter when the pressure in the aspiration catheter is lower than that in the supply tube.

7. A cyclic aspiration catheter comprising:
a first tube having a proximal end and an open distal end;
a second tube having a proximal and an open distal end;
a pulsatile pressure and aspiration source being in fluid communication with the proximal end of the first catheter;
a fluid reservoir being in fluid communication with proximal end of the second tube; and
a first catheter having a proximal end and an open distal end.

8. The cyclic aspiration catheter of claim 7, further comprising a connector having a proximal end and a distal end; the proximal end of the connector being in fluid communication with the distal end of the first tube and the distal end of the second tube; the distal end of the connector being in fluid communication with the proximal end of the first catheter.

9. The cyclic aspiration catheter of claim 8, the connector having a taper section between its proximal end and its distal end.

10. The cyclic aspiration catheter of claim 9, the taper section tapering radially inwardly in the distal direction from a proximal end of the taper section to a distal end of the taper section.

11. The cyclic aspiration catheter of claim 10, the open distal end of the first tube terminating distally of the open distal end of the second catheter.

12. The cyclic aspiration catheter of claim 10, the open distal end of the first tube and the open distal end of the second tube terminate at approximately the same axial location.

13. The cyclic aspiration catheter of claim 11, the open distal end of the first tube and the open distal end of the second tube being disposed within the connector.

14. A method of reducing a risk of air bubbles from entering the vasculature when operation a cyclic aspiration catheter to capture and remove an occlusion within the vessel, the method comprises an aspiration catheter having a proximal end and a distal end; a pulsatile pressure and aspiration source being in fluid communication with the proximal end of the catheter; a supply tube disposed within the aspiration catheter, the supply tube having a proximal end and an open distal end, the open distal end being in fluid communication with the aspiration catheter; and a fluid reservoir being in fluid communication with the supply tube such that a fluid from the reservoir can be drawn into the aspiration catheter, the method comprising:
tracking the aspiration catheter to a site within the vasculature adjacent to an occlusion within the vasculature;
applying an intermittent cyclic interval of vacuum pressure and positive pressure within the aspiration catheter to capture and remove the occlusion;
delivering a liquid from the supply tube within the aspiration catheter during a vacuum pressure portion of the intermittent cyclic interval; and
removing an air bubble from the aspiration catheter via the proximal end of the aspiration catheter during a vacuum pressure portion of the intermittent cyclic interval.

15. The method of reducing a risk of air bubbles from entering the vasculature of claim 14, capturing the occlusion at the distal end of the aspiration catheter while applying a vacuum pressure.

16. The method of reducing a risk of air bubbles from entering the vasculature of claim 15, applying a peak amplitude of the positive pressure that is sufficiently high to push the occlusion in the distal direction in the distal end of the aspiration catheter while maintaining at least a portion of the occlusion within the aspiration catheter.

17. The method of reducing a risk of air bubbles from entering the vasculature of claim 16, permitting liquid within the catheter to escape distally past the occlusion into the vasculature on a positive pressure pulse.

18. The method of reducing a risk of air bubbles from entering the vasculature of claim 17, permitting liquid within the catheter to escape distally past the occlusion into the vasculature on every positive pressure pulse.

19. The method of reducing a risk of air bubbles from entering the vasculature of claims 18, after permitting liquid within the catheter to escape distally past the occlusion into the vasculature on a positive pressure pulse, applying a vacuum pressure to pull the occlusion proximally within the aspiration catheter.

20. The method of reducing a risk of air bubbles from entering the vasculature of claim 19, the delivering a liquid from the supply tube includes delivering the liquid within the aspiration catheter at the open distal end of the supply tube, the distal end of the supply tube being spaced proximally from the distal end of the aspiration catheter by a predetermined distance.
